(19) United States
(12) Patent Application Publication
(10) Pub. No.: US 2026/0061722 A1
(43) Pub. Date: Mar. 5, 2026

(54) APPARATUS AND METHOD FOR PRODUCING ABSORBENT BED PADS, OR ABSORBENT PET MATS

(71) Applicant: GDM S.p.A., Bologna (IT)

(72) Inventors: Alessandro ZAVALLONI, Bologna (IT); Eros ROSSI, Bologna (IT)

(21) Appl. No.: 19/271,876

(22) Filed: Jul. 17, 2025

(30) Foreign Application Priority Data
Sep. 4, 2024 (IT) 102024000019684

(51) Int. Cl. B32B 3/04; A01K 1/01; A01K 1/015; B32B 5/02; B32B 29/02; B32B 37/24; B32B 38/00

(52) U.S. Cl. CPC B32B 3/04; A01K 1/0107; A01K 1/01577; B32B 5/022; B32B 29/02; B32B 37/24; B32B 38/0012; B32B 2262/062; B32B 2264/025; B32B 2305/28; B32B 2307/726; B32B 2317/12; B32B 2317/18; B32B 2571/00

(57) ABSTRACT

An apparatus for producing absorbent bed pads or absorbent pet mats, of the type having an absorbent core with a support layer and a plurality of absorbent fibres arranged on a first surface of the support layer. The apparatus includes a deposition station with a conveyor belt movable along an advancing direction and configured to support a support layer strip, a deposition device configured to deposit the plurality of absorbent fibres on a deposition surface of the support layer strip and a pair of cover members arranged between the deposition device and the conveyor belt and configured to be arranged above opposite longitudinal flaps of the support layer strip. A method that can be carried out by the apparatus.

### FIELD OF THE INVENTION

The present invention refers to an apparatus and a method for producing absorbent bed pads or absorbent pet mats.

### BACKGROUND

Absorbent pads act as a barrier between a user and the bed or other surfaces on which the user stands. Similarly, the absorbent mats act as a barrier between the pet and the litter box or other surfaces on which the pet stands. In the following description reference will be made to the non-limiting example of absorbent bed pads, but what will be described also applies to the absorbent mats.

The absorbent bed pads are articles of assistance for the management of the incontinence. They are designed to absorb any leakage of urine or other body secretions during sleep, keeping the bed dry and comfortable.

Typically, an absorbent bed pad has a substantially rectangular shape and comprises a lower layer (also called “backsheet”), an upper layer (also called “topsheet”) and an absorbent core arranged between the lower layer and the upper layer.

The lower layer is configured to be arranged on the bed and is made of a material impermeable to the body fluids, for example polyethylene or bioplastic.

The upper layer is configured to be arranged in a position contiguous to the user's body and is made of a material that is permeable to the body fluids. This layer is typically made of a soft, breathable material and allows the body fluid to pass through this layer and reach the absorbent core below, keeping the user dry and reducing the risk of irritations.

The absorbent core comprises a support layer and a plurality of absorbent fibres arranged on a surface of the support layer. The absorbent core has the function of absorbing the body fluids that pass through the upper layer and retaining them without leakage even for several hours.

The support layer is generally made of paper.

Typically, the absorbent fibres comprise cellulose and/or one or more super-absorbent polymers (SAP), for example sodium polyacrylate, typically in granular form, which may be confined inside, or within a coating of, cellulose or other similar materials.

The realization of the absorbent bed pads envisages, among other things, supplying a support layer strip onto a conveyor belt and depositing the absorbent fibres on a deposition surface of the support layer strip, over the entire width of the latter.

The deposition of the absorbent fibres takes place by fall from a deposition device arranged above the conveyor belt. The fall of the absorbent fibres is aided by an air flow which, coming from the deposition device, passes through the support layer strip and the conveyor belt.

The deposition device typically has a width greater than that of the support layer strip, so as to be able to deposit the absorbent fibres over the entire width of the latter regardless of the size of the absorbent bed pad that it is wished to be made. The excess absorbent fibres along the width of the support layer strip are collected.

In order to separate in a precise manner the absorbent fibres deposited on the support layer strip from those in excess, it is known to provide, at a downstream end portion of the conveyor belt with reference to its direction of movement and along a transverse direction with respect to the aforesaid direction of movement, two air blowers, each arranged near a respective side of the conveyor belt and, therefore, at a respective longitudinal edge of the support layer strip deposited on the conveyor belt. The air blowers are configured to each generate a respective concentrated air jet that laps the respective longitudinal edge of the support layer strip, thereby forcing the separation of the absorbent fibres in excess from those deposited on the support layer strip.

### SUMMARY

The Applicant has thought about how to separate in a precise manner the absorbent fibres deposited on the support layer strip from those in excess without making use of the aforesaid air blowers and has realised that it is possible to do so by covering the opposite longitudinal flaps of the support layer strip through cover members arranged above the aforesaid longitudinal flaps.

The present invention therefore concerns, in a first aspect thereof, an apparatus for producing absorbent bed pads, or absorbent pet mats, of the type comprising an absorbent core comprising a support layer and a plurality of absorbent fibres arranged on a first surface of the support layer.

Preferably, a deposition station is provided.

Preferably, the deposition station comprises a conveyor belt.

Preferably, the conveyor belt is movable along an advancing direction.

Preferably, the conveyor belt is configured to support a support layer strip.

Preferably, the deposition station comprises a deposition device.

Preferably, the deposition device is configured to deposit the plurality of absorbent fibres on a deposition surface of the support layer strip.

Preferably, the deposition station comprises a pair of cover members.

Preferably, the cover members are arranged between the deposition device and the conveyor belt.

Preferably, the cover members are configured to be arranged above opposite longitudinal flaps of the support layer strip.

The present invention allows to separate with precision, by means of the aforesaid cover members, the absorbent fibres deposited on the deposition surface of the support layer strip from the others (herein referred to as “excess fibres”). This separation can take place optimally because the longitudinal flaps of the support layer strip have been previously covered by respective cover members.

In a second aspect thereof, the present invention concerns a method for producing absorbent bed pads, or absorbent pet mats, of the type comprising an absorbent core comprising a support layer and a plurality of absorbent fibres arranged on a first surface of the support layer.

Preferably, a support layer strip is supplied on a conveyor belt of a deposition station.

Preferably, the deposition station comprises a deposition device.

Preferably, the deposition device is configured to deposit the plurality of absorbent fibres on a deposition surface of the support layer strip.

Preferably, the deposition station comprises a pair of cover members.

Preferably, the cover members are arranged between the deposition device and the conveyor belt above opposite longitudinal flaps of the support layer strip.

Preferably, after having supplied the support layer strip onto the conveyor belt, the plurality of absorbent fibres are deposited onto the deposition surface while the support layer strip is moved along an advancing direction.

The method of the invention can be carried out by employing an apparatus according to the first aspect of the present invention and allows to achieve the same advantages discussed with reference to the apparatus of the invention.

The present invention may have, in both aspects discussed above, at least one of the preferred features described below. These features can therefore be present individually or in combination with each other, unless expressly stated otherwise, both in the apparatus of the first aspect of the present invention and in the method of the second aspect of the present invention.

Preferably, a forming station is provided.

Preferably, the forming station is arranged downstream of the deposition station along an advancing direction of the support layer strip.

Preferably, the forming station comprises folding members.

Preferably, the folding members are configured to fold the opposite longitudinal flaps of the support layer strip over the deposition surface.

This expedient makes it possible to produce a type of absorbent bed pad provided with folds at the opposite longitudinal edges. The folds are obtained by folding the longitudinal flaps of the support layer strip over the deposition surface by means of the folding members. Folding takes place only after the aforesaid separation of the absorbent fibres deposited onto the deposition surface of the support layer strip from the excess fibres has taken place, this separation being obtained by means of the aforesaid cover members.

Preferably, each cover member has a substantially flat surface facing the conveyor belt. Preferably, each cover member further has an inclined surface facing away from the conveyor belt. This inclined surface facilitates the fall of the excess fibres outside the deposition surface, contributing to the achievement of a precise separation between deposited fibres and excess fibres.

Preferably, the cover members can be moved along a transverse direction orthogonal to the advancing direction, so as to be able to make absorbent bed pads of different sizes.

Preferably, the folding members are configured to continuously fold the opposite longitudinal flaps of the support layer strip over the deposition surface while the support layer strip passes through the forming station.

Preferably, the folding members are arranged at opposite sides with respect to the support layer strip when the support layer strip passes through the forming station.

Preferably, the folding members comprise first folding portions/elements arranged on one side with respect to the support layer strip and second folding portions/elements arranged on the opposite side with respect to the support layer strip.

The first and second folding portions/elements are preferably shaped and oriented in such a way as to interfere with the longitudinal flaps of the support layer strip during the movement of the support layer strip in the preparation station and fold them over until reaching the deposition surface, so as to trap some absorbent fibres of the plurality of absorbent fibres between the deposition surface and the respective longitudinal flap of the support layer strip.

Preferably, the folding members are movable along a transverse direction orthogonal to an advancing path of the support layer strip in the forming station. This movement makes it possible to realize absorbent bed pads of different sizes. In particular, it is possible to set the mutual distance between the first folding portions/elements and the second folding portions/elements as a function of the desired deposition surface width.

Preferably, the deposition station comprises a pair of counter-cover members arranged below the conveyor belt at the pair of cover members. The counter-cover members make it possible to avoid creating a vacuum between cover member and longitudinal flap of the support layer strip that could suck up some excess absorbent fibres on the deposition surface, partly nullifying the separation action carried out by the cover members.

Preferably, each counter-cover member has a substantially flat surface facing the conveyor belt.

Preferably, the counter-cover members are movable along a transverse direction orthogonal to the advancing direction, so that they can always be positioned at the cover members.

Preferably, after having deposited the plurality of absorbent fibres onto the deposition surface, the opposite longitudinal flaps of the support layer strip are folded over the deposition surface.

Preferably, the opposite longitudinal flaps of the support layer strip are continuously folded over the deposition surface while the support layer strip is moved along the advancing direction.

### BRIEF DESCRIPTION OF THE DRAWINGS

Further characteristics and advantages of the present invention will become clearer from the following detailed description of a preferred embodiment thereof, made with reference to the appended drawings and provided by way of indicative and non-limiting example, in which:

FIG. 1 is a schematic cross-sectional view of a support layer strip used to produce absorbent bed pads by means of an apparatus in accordance with the present invention, such as that of FIG. 7, this support layer strip being in an initial operating configuration thereof;

FIG. 2 is a schematic cross-sectional view of a portion of the apparatus of FIG. 7 with the support layer strip in the initial operating configuration;

FIG. 3 is a schematic cross-sectional view similar to that of FIG. 2, showing further details of the apparatus of FIG. 7;

FIG. 4 is a schematic cross-sectional view of the support layer strip brought back to its initial operating configuration after absorbent fibres have been deposited thereon;

FIG. 5 is a schematic cross-sectional view of the support layer strip of FIG. 1 in a final operating configuration thereof after the absorbent fibres have been deposited thereon;

FIG. 6 is a schematic cross-sectional view of an absorbent bed pad comprising the support layer strip in its final operating configuration;

FIG. 7 is a schematic side elevational view of an apparatus in accordance with the present invention;

FIG. 8 is a schematic perspective view on an enlarged scale of a member of the apparatus of FIG. 7.

### DETAILED DESCRIPTION

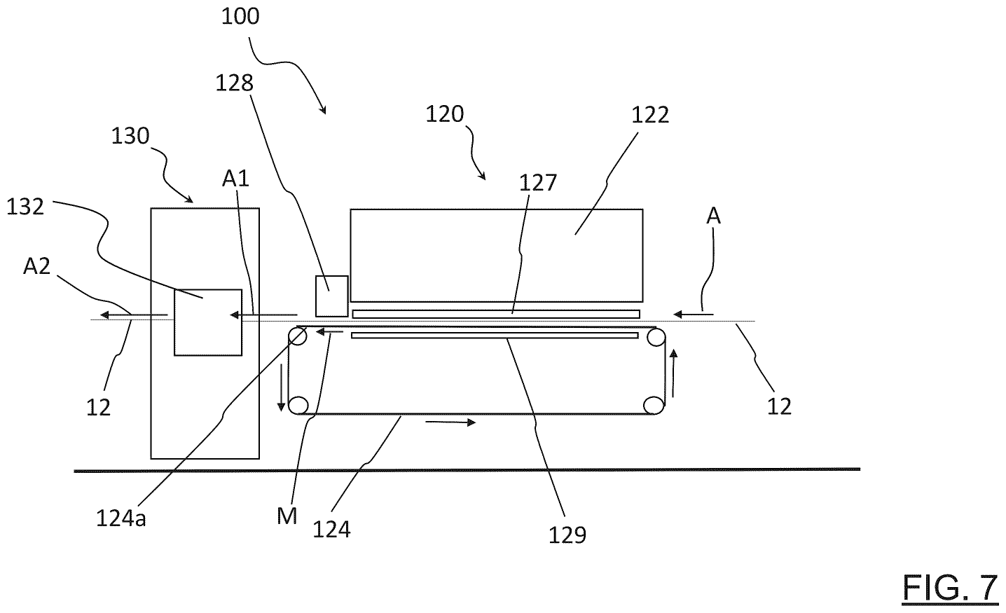

In FIG. 7, the reference numeral 100 is used to indicate an apparatus for producing absorbent bed pads in accordance with the present invention.

Without for this reason losing in generality, explicit reference will be made below to an absorbent pad provided with folds, such as the one illustrated in FIG. 6 and indicated with reference numeral 2. In FIG. 6 the absorbent pad 2 is voluntarily illustrated with the relative components spaced apart from each other, so as to be able to clearly distinguish them. In reality these components are in contact with each other.

The absorbent pad 2 has a substantially rectangular shape and comprises a lower layer 3 (also called “backsheet”), an upper layer 4 (also called “topsheet”) superimposed on the lower layer 3 and an absorbent core 10a arranged between the lower layer 3 and the upper layer 4.

In the following of the present description and in the following claims, the terms “upper”, “lower”, “above”, “below” and the like are used referring to the position assumed by the absorbent pad 2 when it is arranged on a bed (not illustrated), with the lower layer 3 in contact with the bed and the upper layer 4 arranged in such a way as to come into contact with the body of the user.

The lower layer 3 is made of a material impermeable to the body fluids, for example polyethylene, polypropylene or bioplastic, while the upper layer 4 is made of a material permeable to the body fluids, for example non-woven fabric.

The absorbent core 10a comprises a support layer 12a and a plurality of absorbent fibres 20 arranged on a surface 13a of the support layer 12a. The surface 13a faces the upper layer 4.

The support layer 12a may for example be made of paper, while the absorbent fibres 20 comprise cellulose and/or one or more super-absorbent polymers (SAP), for example sodium polyacrylate, typically in granular form, which may be confined inside, or within a coating of, cellulose or other similar materials.

The support layer 12a has opposite side flaps 17a folded over the surface 13a, so that some absorbent fibres 20 are trapped between the surface 13a and the respective side flaps 17a.

The lower layer 3 and the upper layer 4 have a width substantially equal and greater than the width of the absorbent core 10a.

The absorbent pad 2 is produced starting from a support layer strip 12 by means of the apparatus of FIG. 7.

The support layer strip 12 has a deposition surface 13 destined to receive the absorbent fibres 20 and a service surface 15 opposite the deposition surface 13.

The apparatus 100 comprises a deposition station 120 and, in the non-limiting case of production of an absorbent pad provided with folds over, a forming station 130 arranged downstream of the deposition station 120 along an advancing direction A of the support layer strip 12.

The support layer strip 12 is initially supplied to the deposition station 120, from which it is then supplied to the forming station 130.

In particular, the support layer strip 12 is supplied to the deposition station 120 and from this to the forming station 130 with its opposite longitudinal flaps 17 arranged on the same plane of a central longitudinal portion 19 of the support layer strip 12, i.e. with the longitudinal flaps 17 arranged in what in this description is indicated as “initial position”.

The support layer strip 12 subsequently exits the forming station 130 with its opposite longitudinal flaps 17 folded over the deposition surface 13, i.e. with the longitudinal flaps 17 arranged in what in this description is indicated as “final position”.

The deposition station 120 comprises a deposition device 122 configured to deposit the absorbent fibres 20 on the deposition surface 13.

Thus, when the support layer strip 12 passes through the deposition station 120, the deposition surface 13 faces the deposition device 122.

The deposition station 120 also comprises a conveyor belt 124 configured to support the support layer strip 12 and move it along the advancing direction A. The conveyor belt 124 has a width greater than that of the support layer strip 12 and is arranged below the deposition device 122. Further, the conveyor belt 124 is perforated. In FIG. 7 the support layer strip 12 is voluntarily illustrated raised with respect to the conveyor belt 124 to clearly illustrate the support layer strip 12 above the conveyor belt 124. In reality, the support layer strip 12 rests on the conveyor belt 124 at its service surface 15.

The deposition device 122 generates an air flow that facilitates the deposition of the absorbent fibres 20 on the deposition surface 13. The air flow passes through the support layer strip 12 and the conveyor belt 124.

The deposition of the absorbent fibres 20 therefore takes place by fall from the deposition device 122 and by the thrust action exerted by the aforesaid air flow.

The absorbent fibres pass through an opening 122a defined in a lower surface of the deposition device 122. This opening 122a preferably has a width greater than that of the support layer strip 12. As shown in FIG. 3, the absorbent fibres 20 that do not fall onto the support layer strip 12 (herein referred to as “excess fibres”) are sucked in by two suction nozzles 128 arranged above the conveyor belt 124 at opposite sides with respect to the latter and to the advancing direction A, as it will be explained below.

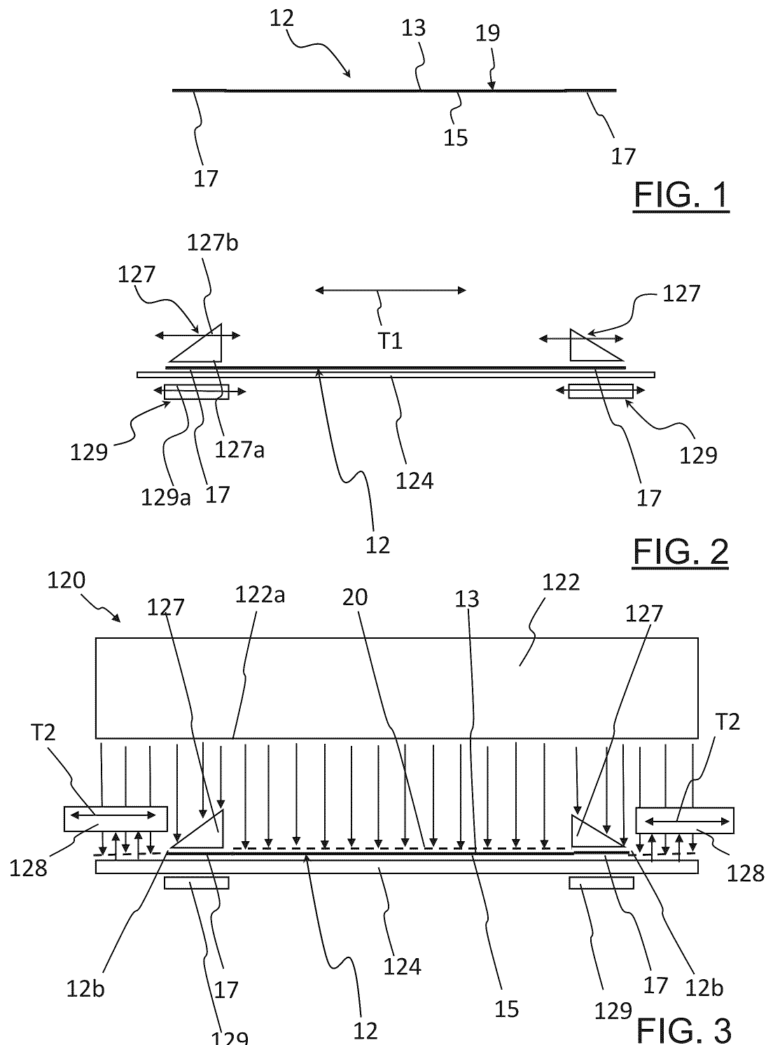

As illustrated in FIGS. 2 and 3, the deposition device 122 further comprises a pair of cover members 127 arranged between the deposition device 120 and the conveyor belt 124, and more precisely between the deposition device 120 and the support layer strip 12.

The cover members 127 are configured to be arranged above the longitudinal flaps 17 of the support layer strip 12, so as to cover them and prevent the deposition thereon of the absorbent fibres 20. The absorbent fibres 20 are then deposited only on the central longitudinal portion 19. Thus, in the deposition station 120 the deposition surface 13 corresponds to the upper surface of the central longitudinal portion 19.

Each cover member 127 has a substantially flat surface 127a facing the conveyor belt 124 and having a width substantially equal to or greater than the width of the longitudinal flaps 17.

The substantially flat surface 127a is arranged near the longitudinal flaps 17. The distance between the substantially flat surface 127a and the respective longitudinal flap 17 of the support layer strip 12 is a few millimetres.

Each cover member 127 also has an inclined surface 127b facing away from the conveyor belt 124. The orientation of the inclined surface 127b is such as to allow the sliding of the absorbent fibres 20 outwards of the respective longitudinal flap 17, outside the support layer strip 12.

In the non-limiting example illustrated in FIGS. 2 and 3, the cover members 127 are rectangular triangle-shaped in cross-section, with a cathetus corresponding to the substantially flat surface 127a and the hypotenuse corresponding to the inclined surface 127b.

The cover members 127 are movable along a transverse direction T1 orthogonal to the advancing direction A so as to be arranged above the longitudinal flaps 17.

As illustrated in FIGS. 2 and 3, the deposition device 122 further comprises a pair of counter-cover members 129 arranged below the conveyor belt 124, each at a respective cover member 127.

Each counter-cover member 129 has a substantially flat surface 129a facing the conveyor belt 124 and having a width substantially equal to the width of the substantially flat surface 127a of the respective cover member 127.

The substantially flat surface 129a is arranged near the conveyor belt 124.

The counter-cover members 129 are movable along the transverse direction T1 similarly to the respective cover members 127, so as to be arranged at the cover members 127.

The two suction nozzles 128 are arranged downstream of the deposition device 122, with reference to the advancing direction A, and upstream of a downstream end portion 124a of the conveyor belt 124, with reference to a movement direction M thereof. The two suction nozzles 128 are positioned along a transverse direction T2 with respect to the movement direction M, each near a respective side of the conveyor belt 124.

The suction nozzles 128 are movable along the transverse direction T2, which is orthogonal to the advancing direction A, so as to be able to be arranged at opposite longitudinal edges 12b of the support layer strip 12.

Each suction nozzle 128 is configured to suck in the excess absorbent fibres 20, i.e. those that following the separation carried out by the cover members 127 are in an external position with respect to these longitudinal edges 12b, from the absorbent fibres 20 that instead are entirely deposited on the central longitudinal portion 19, and therefore on the deposition surface 13. The excess absorbent fibres 20 are then separated from the others and collected before the support layer strip 12 leaves the conveyor belt 124.

The forming station 130 comprises folding members 132 configured to continuously fold the opposite longitudinal flaps 17 of the support layer strip 12 over the deposition surface 13 while the support layer strip 12 passes through the forming station 130 along an advancing path A1.

The folding members 132 can be of conventional type.

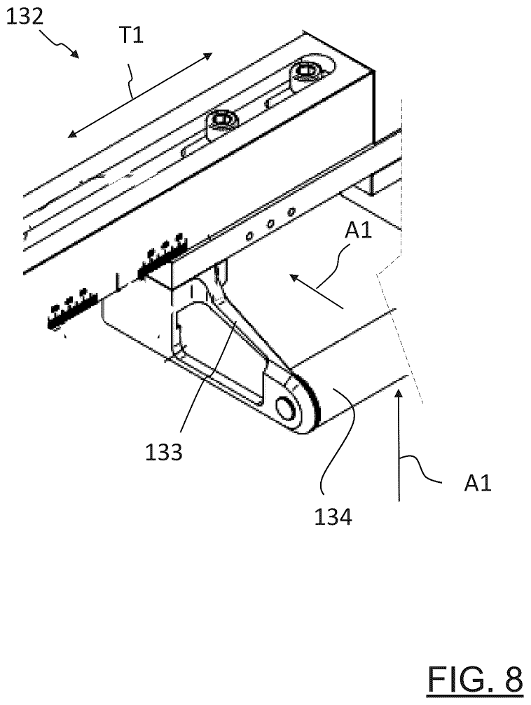

By way of non-limiting example, the folding members 132 can be of the type illustrated in FIG. 8. They comprise folding elements 133 (FIG. 8 shows only one folding element 133) intended to come into contact with the longitudinal flaps 17 of the support layer strip 12 and fold these longitudinal flaps 17 over the deposition surface 13 while the support layer strip 12 passes through the forming station 130 along the advancing path A1 and after the support layer strip 12, supported by a counter roller 134 located upstream of the folding elements 133 along the advancing path A1, is oriented so as to interfere with the folding elements 133.

The folding members 132 are movable along a transverse direction T1 with respect to the advancing path A1 so as to be able to be arranged from time to time in the desired position.

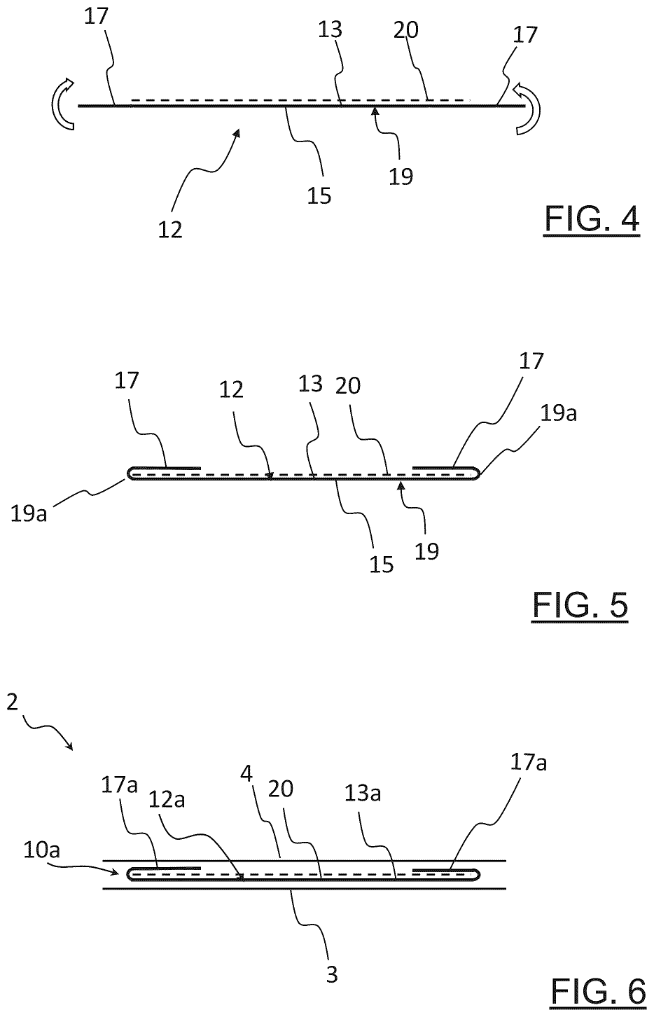

FIGS. 4 and 5 show two operating configurations of the support layer strip 12 assumed in sequence in the forming station 130, and then after the absorbent fibres 20 have been deposited on the deposition surface 13.

In the operating configuration of FIG. 4 the longitudinal flaps 17 are on the same plane of the central longitudinal portion 19, i.e. in their initial position, illustrated in FIG. 1.

The operating configuration of FIG. 5 is assumed after the folding members 132 have folded the longitudinal flaps 17 over the central longitudinal portion 19, i.e. in their final position. In this final position the longitudinal flaps 17 cover the absorbent fibres 20 deposited near opposite longitudinal edges 19a of the central longitudinal portion 19. The longitudinal flaps 17 are folded over at the opposite longitudinal edges 19a (FIG. 5).

The apparatus 100 further comprises, downstream of the forming station 130 along an advancing direction A2 of the support layer strip 12 (which is in the operating configuration of FIG. 5), an application station (not illustrated because of conventional type) configured to apply a strip of lower layer 3 and a strip of upper layer 4 below and above the support layer strip 12, respectively, after the longitudinal flaps 17 have been folded over the deposition surface 13.

Obviously, a person skilled in the art, in order to satisfy specific and contingent needs, can make numerous modifications and variations to the invention described above while remaining within the scope of protection defined by the following claims.

1. An apparatus for producing absorbent bed pads, or absorbent pet mats, of a type comprising an absorbent core comprising a support layer and a plurality of absorbent fibres arranged on a first surface of said support layer, said apparatus comprising a deposition station comprising a conveyor belt movable along an advancing direction (A) and configured to support a support layer strip, a deposition device configured to deposit said plurality of absorbent fibres on a deposition surface of said support layer strip and a pair of cover members arranged between the deposition device and the conveyor belt and configured to be arranged above opposite longitudinal flaps of the support layer strip.

2. The apparatus according to claim 1, further comprising a forming station arranged downstream of the deposition station along an advancing direction (A) of said support layer strip and comprising folding members configured to fold the opposite longitudinal flaps of the support layer strip over said deposition surface.

3. The apparatus according to claim 1, wherein each cover member has a substantially flat surface facing the conveyor belt and an inclined surface facing away from the conveyor belt.

4. The apparatus according to claim 1, wherein the cover members are movable along a transverse direction (T) orthogonal to the advancing direction (A).

5. The apparatus according to claim 2, wherein the folding members are configured to continuously fold the opposite longitudinal flaps of the support layer strip over said deposition surface while the support layer strip passes through the forming station.

6. The apparatus according to claim 5, wherein the folding members are movable along a transverse direction (T) orthogonal to an advancing path of the support layer strip in the forming station.

7. The apparatus according to claim 1, wherein the deposition station comprises a pair of counter-cover members arranged below the conveyor belt at said pair of cover members.

8. The apparatus according to claim 7, wherein each counter-cover member has a substantially flat surface facing the conveyor belt.

9. The apparatus according to claim 7, wherein the counter-cover members are movable along a transverse direction (T) orthogonal to said advancing direction (A).

10. A method for producing absorbent bed pads, or absorbent pet mats, of a type comprising an absorbent core comprising a support layer and a plurality of absorbent fibres arranged on a first surface of said support layer, said method comprising in sequence:
supplying a support layer strip onto a conveyor belt of a deposition station comprising a deposition device, configured to deposit said plurality of absorbent fibres onto a deposition surface of said support layer strip, and a pair of cover members arranged between the deposition device and the conveyor belt above opposite longitudinal flaps of the support layer strip;
depositing said plurality of absorbent fibres on said deposition surface while said support layer strip is moved along an advancing direction (A).

11. The method according to claim 10, further comprising folding the opposite longitudinal flaps of the support layer strip over said deposition surface.

12. The method according to claim 11, wherein the opposite longitudinal flaps of the support layer strip are continuously folded over said deposition surface while the support layer strip is moved along the advancing direction (A).
